(19) United States
(12) Patent Application Publication
(10) Pub. No.: US 2004/0158112 A1
(43) Pub. Date: Aug. 12, 2004

(54) Silicon carbide-supported catalysts for oxidative dehydrogenation of hydrocarbons

(75) Inventors: Sriram Ramani, Ponca City, OK (US); Joe D. Allison, Ponca City, OK (US); Lisa M. Carmichael, Ponca City, OK (US); Zhen Chen, Ponca City, OK (US)

Correspondence Address: KIM S. MANSON, CONOCOPHILIPS COMPANY, P.O. BOX 4783, HOUSTON, TX, 77210-4783, US

(73) Assignee: ConocoPhillips Company, Houston, TX

(21) Appl. No.: 10/364,177

(22) Filed: Feb. 10, 2003

(51) Int. Cl. C07C 5/373; C07C 5/327; B01J 27/224

(52) U.S. Cl. 585/658; 502/178; 502/182; 502/185

(57) ABSTRACT

A catalyst useful for the production of olefins from alkanes via oxidative dehydrogenation (ODH) is disclosed. The catalyst includes a silicon carbide support. The catalyst may optionally include a base metal, metal oxide, or combination thereof. A base metal is herein defined as a non-Group VIII metal, with the exception of iron, cobalt and nickel. Suitable base metals include Group IB-VIIB metals, Group IIIA-VA metals, Lanthanide metals, iron, cobalt and nickel. Suitable metal oxides include alumina, stabilized aluminas, zirconia, stabilized zirconias (PSZ), titania, ytteria, silica, niobia, and vanadia. Additionally, the catalyst may optionally include a Group VIII promoter. Suitable Group VIII promoters include Ru, Rh, Pd, Os, Ir, and Pt.

### FIELD OF THE INVENTION

This invention relates to silicon carbide-supported catalyst compositions for oxidative dehydrogenation processes and a method of using such catalysts in the presence of hydrocarbons. More particularly this invention relates to the compositions of silicon carbide-supported catalysts for the production of olefins by oxidative dehydrogenation of hydrocarbons in short-contact time reactors (SCTRs).

### BACKGROUND OF THE INVENTION

Dehydrogenation of hydrocarbons is an important commercial process. Dehydrogenation is the process used to convert aliphatics to olefins, mono-olefins to di-olefins, cycloalkanes to aromatics, alcohols to aldehydes and ketones, aliphatics and olefins to oxygenates, etc., by removing hydrogen chemically. In more practical terms, this process is responsible for products such as detergents, gasolines, pharmaceuticals, plastics, polymers, synthetic rubbers and many others. In addition, there is significant commercial use of the process for making many of the precursors for the above mentioned products. For example, polyethylene is made from ethylene, which is made from the dehydrogenation of ethane (i.e. aliphatic to olefin). More ethylene is produced in the U.S. than any other organic chemical. Thus, it is easy to appreciate the significance of the dehydrogenation process to industry.

Traditionally, the dehydrogenation of hydrocarbons has been carried out using steam cracking or non-oxidative dehydrogenation processes. Thermal or steam cracking is an extremely energy intensive process that requires temperatures in excess of 800° C. About 1.4×10¹⁵ BTU's (equivalent to burning 1.6 trillion ft³ of natural gas) are consumed annually to produce ethylene. In addition, much of the reactant (ethane) is lost as coke deposition. Non-oxidative dehydrogenation is dehydrogenation whereby no molecular oxygen is added.

Oxidative dehydrogenation of hydrocarbons (ODH) with short contact time reactors is an alternative to traditional steam cracking and non-oxidative dehydrogenation processes. During short contact time ODH reactions, oxygen is co-fed with saturated hydrocarbons to form a reactant gas which then has a catalyst contact time of typically <10 milliseconds. The oxygen may be fed as pure oxygen, air, oxygen-enriched air, oxygen mixed with a diluent, and so forth. Oxygen in the desired amount may be added in the feed to the dehydrogenation zone and oxygen may also be added in increments to the dehydrogenation zone. At 5 psig pressure with monolith-supported catalysts, the reaction temperature is typically between 800-1100° C.

The capital costs for olefin production via ODH are significantly less than with the traditional processes, because ODH uses simple fixed bed reactor designs and high volume throughput. In addition, ODH is an autothermal process, which requires no or very little energy to initiate the reaction. Energy savings over traditional, endothermal processes can be significant if the heat produced with ODH is recaptured and recycled. Generally, the trade-off for saving money in commercial processes is loss of yield or selectivity; however, the ODH reactions are comparable in selectivity and conversion to steam cracking.

The benefits of ODH are not new. ODH processes have been studied on the laboratory scale for some time. The current methods in ODH reactions involve the use of platinum-and-chromium containing catalysts.

Platinum and chromium oxide-based monolith catalysts were used for ethylene production with SCTRs in U.S. Pat. No. 6,072,097 and WO Pub. No. 00/43336, respectively. The monoliths used in these catalysts were ceramic domes with 20-100 pores per linear inch. The domes were comprised of Al₂O₃, SiO₂, Mg-stabilized ZrO₂ (PSZ) or Y-stabilized ZrO₂ (YSZ). Ethylene yield with these reactors was about 50-55%.

U.S. Pat. No. 6,072,097 describes the use of Pt-coated monolith catalysts for ODH reactions in SCTRs. Pt in the range of 0.2-10% total weight of catalyst was claimed effective for ODH. Further impregnation of Sn or Cu on the Pt-coated surface (at Sn:Pt or Cu:Pt ratios of 0.5:1-7:1) promoted the ODH reactions. The light-off temperature with this type of catalysts was about 220° C., with reduced or no preheat after the light-off procedure. Light-off temperature is herein defined as the minimum temperature of the gases entering the catalyst zone at which the catalyst reaches a chemically active state so as to initiate a self-sustaining reaction between hydrocarbon(s) and oxygen (or oxygen-containing gas), as indicated by an increase in the temperature of the gases exiting the catalyst zone. The disadvantage of using Pt-based catalysts is the high cost of Pt.

WO Patent No. 0043336 describes the use of Cr, Cu, Mn or their mixed oxide-loaded monolith as the primary ODH catalysts promoted with less than 0.1% Pt. In addition, small amounts of Mn, Mg, Ni, Fe and Ag were used as promoters. Light-off temperature with these catalysts was about 350° C., with or without reduced preheat after the light-off procedure. Comparable ethylene yields to those in U.S. Pat. No. 6,072,097 were obtained.

As can be seen, of the methods that employ catalysts for oxidative dehydrogenation of hydrocarbons to olefins, typically catalytic metals are dispersed throughout a ceramic oxide support. Ceramics oxides however, are known to have relatively low thermal conductivities. This poses a problem because the formation of hot spots, in which the temperature is higher than in the remaining part of the catalyst bed, can occur. These hot spots give rise to secondary reactions such as the total combustion of the starting material or lead to the formation of undesired by-products, which can be separated from the reaction product only with great difficulty, if at all. In addition, formation of secondary products decreases the overall efficiency of the desired process, and leads to significant increase in costs.

Accordingly, there is a continuing need for better, more economical processes and catalysts for the oxidative dehydrogenation of hydrocarbons, in which the catalyst retains a high level of activity and selectivity to olefins under conditions of high gas space velocity and elevated pressure.

### SUMMARY OF THE INVENTION

In order to operate at very high flow rates, high pressure and using short contact time CPOX reactors, the catalysts should be highly active, have excellent mechanical strength, resistance to rapid temperature fluctuations and thermal stability at oxidative dehydrogenation reaction temperatures.

The catalysts and methods of the present invention overcome some of the drawbacks of existing catalysts and processes for converting light hydrocarbons to olefins. The new silicon carbide-supported catalysts may demonstrate greater thermal stability than ceramic oxide-supported catalysts and give comparable olefin yield to conventional oxidative dehydrogenation catalysts under conditions of high gas space velocity and elevated pressure. Another advantage provided by the preferred new catalysts and processes is that they are economically feasible for use under commercial-scale conditions with little or no increase in capital cost.

The present invention provides a catalyst system for use in ODH that allows high conversion of the hydrocarbon feedstock at high gas hourly space velocities, while maintaining high selectivity of the process to the desired products. For the purposes of this disclosure, all listed metals are identified using the CAS naming convention.

In accordance with a preferred embodiment of the present invention, a catalyst for use in ODH processes includes a silicon carbide support. The catalyst may optionally include a base metal, metal oxide, or combination thereof. A base metal is herein defined as a non-Group VIII metal, with the exception of iron, cobalt and nickel. Suitable base metals include Group IB-VIIB metals, Group IIIA-VA metals, Lanthanide metals, iron, cobalt and nickel. Suitable metal oxides include alumina, stabilized aluminas, zirconia, stabilized zirconias (PSZ), titania, ytteria, silica, niobia, and vanadia. Additionally, the catalyst may optionally include a Group VIII promoter. Suitable Group VIII promoters include Ru, Rh, Pd, Os, Ir, and Pt.

In accordance with another preferred embodiment of the present invention, a method for the production of olefins includes contacting a preheated alkane and oxygen stream with a silicon carbide-supported catalyst, sufficient to initiate the oxidative dehydrogenation of the alkane (the preheat temperature being between 75° C. and 800° C.), maintaining a contact time of the alkane with the catalyst for less than 100 milliseconds, and maintaining oxidative dehydrogenation favorable conditions.

These and other embodiments, features and advantages of the present invention will become apparent with reference to the following description.

### DETAILED DESCRIPTION OF THE PREFERRED EMBODIMENTS

A new family of oxidative dehydrogenation catalysts having a silicon carbide support is described in the following representative examples. These catalysts are capable of catalytically converting C₂-C₁₀ hydrocarbons to olefins. The inventors demonstrate that new silicon carbide-supported catalysts, when prepared as described in the following examples, are highly active oxidative dehydrogenation catalysts with sufficient mechanical strength to withstand high pressures and temperatures and permit a high flow rate of reactant and product gases when employed on-stream in a short contact time reactor for olefin production.

Without wishing to be restricted to a particular theory, the inventors believe that the high thermal conductivity of the silicon carbide support serves to minimize the number of hot spots, which in turn, serves to limit secondary reactions (i.e. combustion), while maintaining sufficient crush strength. Crush strength, also known as mechanical strength, is herein defined as the load at which the support physically breaks. Because silicon carbide dissipates the heat formed during oxidative dehydrogenation, secondary reactions are prevented from equilibrating. This results in a higher product selectivity, or a more selective catalyst. Additionally, by maintaining a lower temperature in the system, the amount of catalytically active metals volatilizing may be reduced.

As is known, silicon carbide (SiC) is composed of tetrahedra of carbon and silicon atoms with strong bonds in the crystal lattice. These strong bonds produce a very tough material. For example, SiC is not attacked by any acids or alkalis or molten salts up to 800° C. In air, SiC forms a protective silicon oxide coating at 1200° C. and can be used up to 1600° C. The high thermal conductivity coupled with low thermal expansion and high strength give SiC exceptional thermal shock resistant qualities.

Key properties of SiC include high strength, low thermal expansion, high thermal conductivity, high hardness, excellent thermal shock resistance, and superior chemical inertness. In addition, SiC has a very high decomposition temperature (>2000° C.) and has long-term stability in oxidizing atmospheres up to temperatures above 1400° C.

Catalyst System

It will be understood that the selection of a catalyst or catalyst system requires many technical and economic considerations. Key catalyst properties include high activity, high selectivity, high recycle capability and filterability. Catalyst performance is determined mainly by the active metal components. For example, a catalyst might be chosen based both on its ability to complete the desired reaction and its inability to complete an unwanted reaction. Suitable base metals, metal oxides, and combinations thereof, known to aid in lowering the light-off temperature of the ODH reaction, including Group IB-VIIB metals, Group IIIA-VA metals, Lanthanide metals, iron, cobalt, nickel, alumina, stabilized aluminas, zirconia, stabilized zirconias (SZ), titania, ytteria, silica, niobia, vanadia, and any combinations thereof, may be used to coat the supports of the present invention. Additionally, the support may contain promoters that enhance catalyst selectivity and performance, and aid in lowering the light-off temperature of the ODH reaction. Suitable promoters may include, for example Group VIII promoters, including Ru, Rh, Pd, Os, Ir, and Pt, and any combinations thereof. In a preferred embodiment, the promoter is Pd or Pt. It is believed that base metals, metal oxides, and combinations thereof, may improve the promoters' dispersion on the support.

The present catalysts are preferably provided in the form of foam, monolith, gauze, distinct structures, or irregularly shaped particles, for operation at the desired high gas velocities with minimal back pressure. The terms “distinct” or “discrete” structures or particulates, as used herein, refer to supports in the form of divided materials such as granules, beads, pills, pellets, cylinders, trilobes, extrudates, spheres or other rounded shapes, or another manufactured configuration. Alternatively, the divided material may be in the form of irregularly shaped particles. Preferably at least a majority (i.e., >50%) of the particles or distinct structures have a maximum characteristic length (i.e., longest dimension) of less than six millimeters, preferably less than three millimeters.

In a preferred embodiment, the catalysts are provided in the form of pills with a metal loading of approximately 0.1-0.5% Pt or Pd. In an alternate preferred embodiment, the catalysts are provided in the form of monoliths with a metal loading of less than 0.1% Pt or Pd.

Use of high space velocities and millisecond contact times for the commercial scale conversion of light alkanes to corresponding alkenes will reduce capital investment and increase alkene production significantly. It has been discovered that ethylene yield of 55% or higher in a single pass through the catalyst bed is achievable. This technology has the potential to achieve yields above that of the conventional technology at a much lower cost. The need for steam addition, as is currently required in the conventional cracking technology, is also eliminated by the present process. However, in some embodiments, the use of steam may be preferred. There is minimal coking in the present process and therefore little unit down time and loss of valuable hydrocarbon feedstock. Furthermore, the present novel catalysts improve the selectivity of the process to the desired alkene.

In some embodiments, ODH is carried out using the hydrocarbon feed mixed with an appropriate oxidant and possibly steam. Appropriate oxidants may include, but are not limited to air, oxygen-enriched air, I₂, O₂, N₂O and SO₂. Use of the oxidant prevents coke deposition and aids in maintaining the reaction. Steam, on the other hand, may be used to activate the catalyst, remove coke from the catalyst, or serve as a diluent for temperature control.

### EXAMPLES

In the following examples, the silicon carbide foam supports were purchased from Porvair Advanced Materials. In a first layer, the base metal, metal oxide, and base metal-metal oxide coatings were added by an incipient wetness technique, wherein incipient wetness of the supports was achieved using aqueous solutions of a soluble metal salts such as nitrate, acetate, chlorides, acetylacetonate or the like. In a second layer, the Group VIII promoter metals were similarly added by an incipient wetness technique.

While the following examples were prepared by an incipient wetness technique, any technique known to those skilled in the art may be alternatively used. For higher metal loading, the process may be repeated until desired loading is achieved. In addition, in some of the examples, the catalysts contain only a first layer, which was either a base metal, metal oxide, and base metal-metal oxide coating, or a Group VIII promoter coating. The final catalysts tested were in the form of foam monoliths of 20-pores per inch density, and pills of 12-mesh and 20-mesh particle size.

Test Procedure and Results

The following data were collected at total flow rate of 3-5 SLPM, with a fuel-to-oxygen ratio of 1.8. Metal compositions were supported on a 20ppi (pores per linear inch) SiC monolith (Examples 1-7) and on 20-mesh SiC pills (Examples 8-11). Results are shown in Table 3.

TABLE 3

| | | | | | | | | | |
|---|---|---|---|---|---|---|---|---|---|
| | | | Fuel to | Preheat | Catalyst | % | % | % | % |
| | | GHSV | Oxygen | temp. | temp. | Ethane | Oxygen | C₂H₄ | C₂H₄ |
| Ex. | Catalyst | hr-1 | molar ratio | (° C.) | (° C.) | Conv. | Conv. | selectivity | yield |

Foam

| | | | | | | | | | |
|---|---|---|---|---|---|---|---|---|---|
| 1 | No catalyst | 139,860 | 1.8 | 600 | 918 | 54.2 | 63.0 | 40.8 | 22.1 |
| 2 | 0.1Rh | 139,860 | 1.8 | 300 | 896 | 87.4 | 96.8 | 55.4 | 48.4 |
| 3 | 0.1Rh/1.7Sn | 139,860 | 1.8 | 305 | 912 | 90.7 | 98.1 | 56.0 | 50.7 |
| 4 | 0.1Rh/1.6Mn | 139,860 | 1.8 | 300 | 866 | 86.2 | 97.5 | 52.9 | 45.6 |
| 5 | 0.1Pt/2.0Cr | 139,860 | 1.8 | 350 | 938 | 88.6 | 96.9 | 53.7 | 47.6 |
| 6 | 0.1Pt/1.8Mn | 139,860 | 1.8 | 350 | 970 | 94.1 | 98.4 | 54.2 | 51.0 |
| 7 | 0.1Ru/1.6Cr | 139,860 | 1.8 | 296 | 902 | 82.2 | 97.2 | 49.6 | 40.7 |

Pills

| | | | | | | | | | |
|---|---|---|---|---|---|---|---|---|---|
| 8 | 0.5Pt | 111,890 | 1.8 | 300 | 837 | 79.5 | 97.1 | 66.3 | 52.7 |
| | | 186,470 | 1.8 | 300 | 906 | 83.7 | 97.5 | 65.4 | 54.7 |
| 9 | 0.5Pt/2Sn | 111,890 | 1.8 | 300 | 843 | 85.8 | 98.9 | 67.2 | 57.6 |
| | | 186,470 | 1.8 | 300 | 900 | 86.5 | 98.4 | 65.7 | 56.8 |
| 10 | 2Pt | 111,890 | 1.8 | 300 | 846 | 82.2 | 97.6 | 66.5 | 54.7 |
| | | 186,470 | 1.8 | 300 | 909 | 85.2 | 97.7 | 65.3 | 55.7 |
| 11 | 2Pt/2Sn | 111,890 | 1.8 | 300 | 838 | 84.7 | 99.0 | 67.4 | 57.1 |
| | | 186,470 | 1.8 | 300 | 895 | 86.3 | 98.5 | 66.5 | 57.4 |

From the results, it can be seen that silicon carbide foam without any catalyst requires a preheat temperature of approximately 600° C. and achieves approximately 22% ethylene yield. adding 0.1% Rh to the silicon carbide foam support lowers the preheat temperature to approximately 300° C. and increases the ethylene yield to approximately 48%.

Examining the results obtained using particulate SiC support, it can be seen that the ODH performance improved through better ethylene selectivity. It is interesting that the ethylene conversion and yield generally increased as the flowrate increased, indicating that the shorter contact time is preferred for the ODH process by minimizing secondary reactions. As high as 57.6% ethylene yield was achieved using a 0.5% Pt/2% Sn on 20-mesh SiC catalyst. It must be mentioned that this result was achieved without any optimization of the process conditions, e.g., flowrate, fuel/oxygen ratio, catalyst packing, etc. It is believed that with further optimization, significant improvement in olefin yield can be attained using this catalyst support.

Process Conditions

Any suitable reaction regime is applied in order to contact the reactants with the catalyst. One suitable regime is a fixed bed reaction regime, in which the catalyst is retained within a reaction zone in a fixed arrangement. Catalysts may be employed in the fixed bed regime; retained using fixed bed reaction techniques well known in the art. Several schemes for carrying out catalytic partial oxidation (CPOX) of hydrocarbons in a short contact time reactor have been described in the literature and one of ordinary skill in the art will understand the operation of short contact time reactors and the applicability of the present invention thereto.

Accordingly, a feed stream comprising a hydrocarbon feedstock and an oxygen-containing gas is contacted with one of the above-described catalysts in a reaction zone maintained at conversion-promoting conditions effective to produce an effluent stream comprising alkenes. The hydrocarbon feedstock may be any gaseous hydrocarbon having a low boiling point, such as ethane, natural gas, associated gas, or other sources of light hydrocarbons having from 2 to 10 carbon atoms. In addition, hydrocarbon feeds including naphtha and similar feeds may be employed. The hydrocarbon feedstock may be a gas arising from naturally occurring reserves of ethane. Preferably, the feed comprises at least 50% by volume alkanes (<C₁₀).

The hydrocarbon feedstock is contacted with the catalyst as a gaseous phase mixture with an oxygen-containing gas, preferably pure oxygen. The oxygen-containing gas may also comprise steam and/or methane in addition to oxygen. Alternatively, the hydrocarbon feedstock is contacted with the catalyst as a mixture with a gas comprising steam and/or methane.

The process is operated at atmospheric or superatmospheric pressures, the latter being preferred. The pressures may be from about 80 kPa to about 32,500 kPa, preferably from about 130 kPa to about 3,500 kPa. The preheat temperature of the present invention occurs at temperatures of from about 75° C. to about 800° C., preferably from about 150° C. to about 700° C. when a silicon carbide support without metal loading is used. The preheat temperature of the present invention occurs at temperatures of from about 150° C. to about 700° C., preferably from about 150° C. to about 500° C. when a silicon carbide support with metal loading is used. The hydrocarbon feedstock and the oxygen-containing gas are preferably pre-heated before contact with the catalyst. The hydrocarbon feedstock and the oxygen-containing gas are passed over the catalyst at any of a variety of space velocities.

Gas hourly space velocities (GHSV) for the present process, stated as normal liters of gas per liters of catalyst per hour, are from about 20,000 to at least about 100,000,000 hr⁻¹, preferably from about 50,000 to about 1,000,000 hr⁻¹. The process preferably includes maintaining a catalyst residence time of no more than 100 milliseconds for the reactant gas mixture. An effluent stream of product gases, including alkenes, CO, CO₂, H₂, H₂O, and unconverted alkanes emerges from the reactor.

In some embodiments, unconverted alkanes may be separated from the effluent stream of product gases and recycled back into the feed.

In some embodiments the use of steam may be employed. As mentioned above, steam may be used to activate the catalyst, remove coke from the catalyst, or serve as a diluent for temperature control.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. For example, the present invention may be incorporated into a gas to liquids plant (GTL) or may stand alone. Accordingly, the scope of protection is not limited by the description set out above, but is only limited by the claims which follow, that scope including all equivalents of the subject matter of the claims. The disclosures of all patents and publications cited herein are incorporated by reference in their entireties.

What is claimed is:

1. A method for converting gaseous hydrocarbons to olefins comprising:
heating a feed stream comprising an alkane and an oxidant to a temperature of approximately 75° C. to 800° C.;
contacting the feed stream with a catalyst comprising a silicon carbide support;
maintaining a contact time of the alkane with the catalyst for less than 100 milliseconds; and
maintaining oxidative dehydrogenation favorable conditions.

2. The method of claim 1 wherein the oxidant is an oxygen-containing gas.

3. The method of claim 1 wherein the oxidant is essentially pure oxygen.

4. The method of claim 1 wherein the feed stream is heated to a temperature less than about 600° C.

5. The method of claim 1 wherein the ethylene yield is at least 25%.

6. The method of claim 1 wherein the ethylene yield is at least 40%.

7. The method of claim 1 wherein the support comprises at least one monolith.

8. The method of claim 1 wherein the support comprises a plurality of discrete structures.

9. The method of claim 1, further comprising a base metal selected from the group consisting of Group IB-VIIB metals, Group IIIA-VA metals, lanthanide metals, iron, cobalt or nickel, or a metal oxide selected from the group consisting of alumina, stabilized aluminas, zirconia, stabilized zirconias, titania, ytteria, silica, niobia, and vanadia or a combination thereof, wherein the base metal, metal oxide, or a combination thereof is deposited on the silicon carbide support.

10. The method of claim 1, further comprising a promoter metal selected from the group consisting of Ru, Rh, Pd, Pt, Os, and Ir, wherein the promoter metal is deposited on the silicon carbide support.

11. An oxidative dehydrogenation catalyst comprising a silicon carbide support.

12. The catalyst of claim 11 wherein the support comprises at least one monolith.

13. The catalyst of claim 11 wherein the support comprises a plurality of discrete structures.

14. The catalyst of claim 13 wherein the discrete structures are particulates.

15. The catalyst of claim 14 wherein the plurality of discrete structures comprises at least one geometry chosen from the group consisting of powders, particles, granules, spheres, beads, pills, pellets, balls, noodles, cylinders, extrudates and trilobes.

16. The catalyst of claim 13 wherein at least a majority of the discrete structures each have a maximum characteristic length of less than six millimeters.

17. The catalyst of claim 16 wherein the majority of the discrete structures each have a maximum characteristic length of less than three millimeters.

18. The catalyst of claim 11 wherein the ethylene yield is at least 25%.

19. The catalyst of claim 11 wherein the ethylene yield is at least 40%.

20. The catalyst of claim 11 wherein the ethylene yield is at least 50%.

21. The catalyst of claim 11, further comprising a base metal selected from the group consisting of Group IB-VIIB metals, Group IIIA-VA metals, lanthanide metals, iron, cobalt or nickel, or a metal oxide selected from the group consisting of alumina, stabilized aluminas, zirconia, stabilized zirconias, titania, ytteria, silica, niobia, and vanadia or a combination thereof, wherein the base metal, metal oxide, or a combination thereof is deposited on the silicon carbide support.

22. The catalyst of claim 21 wherein the support comprises at least one monolith.

23. The catalyst of claim 21 wherein the support comprises a plurality of discrete structures.

24. The catalyst of claim 23 wherein the discrete structures are particulates.

25. The catalyst of claim 24 wherein the plurality of discrete structures comprises at least one geometry chosen from the group consisting of powders, particles, granules, spheres, beads, pills, pellets, balls, noodles, cylinders, extrudates and trilobes.

26. The catalyst of claim 23 wherein at least a majority of the discrete structures each have a maximum characteristic length of less than six millimeters.

27. The catalyst of claim 26 wherein the majority of the discrete structures each have a maximum characteristic length of less than three millimeters.

28. The catalyst of claim 21 wherein the ethylene yield is at least 50%.

29. The catalyst of claim 21 wherein the preheat temperature is below 400° C.

30. The catalyst of claim 11, further comprising a promoter metal selected from the group consisting of Ru, Rh, Pd, Pt, Os, and Ir, wherein the promoter metal is deposited on the silicon carbide support.

31. The catalyst of claim 30 wherein the promoter metal loading is 0.5% or less of the total weight of the catalyst.

32. The catalyst of claim 30 wherein the promoter metal is Pt or Pd.

33. The catalyst of claim 30 wherein the support comprises a plurality of discrete structures.

34. The catalyst of claim 30 wherein the promoter metal loading is approximately 0.1-0.5% the total weight of the catalyst.

35. The catalyst of claim 30 wherein the ethylene yield is at least 50%.

36. The catalyst of claim 30 wherein the preheat temperature is below 600° C.

37. An oxidative dehydrogenation process catalyst capable of producing an ethylene yield of at least 50% when used to catalyze a conversion of gaseous hydrocarbons to olefins, said catalyst comprising:
a silicon carbide support;
a catalytic material comprising a base metal selected from the group consisting of Group IB-VIIB metals, Group IIIA-VA metals, lanthanide metals, iron, cobalt or nickel, or a metal oxide selected from the group consisting of alumina, stabilized aluminas, zirconia, stabilized zirconias, titania, ytteria, silica, niobia, and vanadia or a combination thereof; and
a promoter metal selected from the group consisting of Ru, Rh, Pd, Pt, Os, and Ir;
wherein said catalytic material and said promoter metal are supported on said support.

38. The catalyst of claim 37 wherein the support comprises at least one monolith.

39. The catalyst of claim 37 wherein the support comprises a plurality of discrete structures.

40. The catalyst of claim 37 wherein the ethylene yield is at least 50%.

41. The catalyst of claim 40 wherein the promoter metal loading is 0.5% or less of the total weight of the catalyst.

42. The catalyst of claim 40 wherein the promoter metal is Pt or Pd.
